(19) United States
(12) Patent Application Publication
(10) Pub. No.: US 2022/0409428 A1
(43) Pub. Date: Dec. 29, 2022

(54) METHOD OF PERFORMING CRYONEUROLYSIS

(71) Applicant: Black Cat Medical LLC, Fortville, IN (US)

(72) Inventor: Michael F. Guzman, Fortville, IN (US)

(73) Assignee: Black Cat Medical LLC, Fortville, IN (US)

(21) Appl. No.: 17/847,310

(22) Filed: Jun. 23, 2022

Related U.S. Application Data
(60) Provisional application No. 63/213,861, filed on Jun. 23, 2021.
(60) Provisional application No. 63/235,217, filed on Aug. 20, 2021.

(51) Int. Cl. A61F 7/12; A61B 17/34; A61M 19/00

(52) U.S. Cl. CPC A61F 7/12; A61B 17/3403; A61M 19/00; A61B 17/3421; A61F 2007/126; A61B 2090/3983

(57) ABSTRACT

Provided are devices and methods for anesthetizing a patient undergoing surgery, and/or pain block procedures. In certain embodiments the disclosure provides a curved cryoneurolysis needle. In some forms, the methods include inserting at least one cryoneurolysis needle into a target region of the patient, and cooling the cryo-needle to inhibit the target intercostal nerve.

### REFERENCE TO RELATED CASES

This application claims the benefit of U.S. Provisional Patent Application No. 63/213,861, filed Jun. 23, 2021, and U.S. Provisional Patent Application No. 63/235,217, filed Aug. 20, 2021, which are both incorporated by reference herein in their entirety.

### BACKGROUND

This present disclosure relates to targeted blockade of nerve impulses. Cryoneurolysis is the reversible inhibition of the signal carrying parts of a nerve, temporarily blocking pain sensation. Cryoneurolysis outcomes are dependent on precise anatomic knowledge and placement of a cryoneurolysis “freeze ball” to a precise location near a target nerve. Intercostal nerve blocks were initially described around 1905, however this block was not widely utilized because of the difficulty in accessing the intercostal nerves which are in close proximity to the lung. A need therefore exists for a device and method which facilitate accurate and optimal placement of a cryoneurolysis needle.

### SUMMARY

In certain aspects, the present disclosure pertains to systems and methods for administering anesthesia and/or pain blocks to a patient. In accordance with some forms, the present disclosure provides cryoneurolysis devices comprising a curved needle. The curved needle having a proximal portion and a distal portion and configured to produce a cold zone at or near the distalmost tip of the curved needle for cryoneurolysis of a target nerve. In some forms, the curve needle comprises a curved portion between the proximal portion and the distal portion. In certain embodiments the proximal portion and/or the distal portion is/are linear. In accordance with some forms the curved needle has an arcuate shape extending from the proximal portion to the distal portion. The cryoneurolysis device may have one or more imagable portions, which may be positioned at or near the distal tip. In accordance with some forms, the curved needle is curved to create an angle between the proximal portion and the distal portion of 45 degrees to 90 degrees.

The present disclosure also provides methods of anesthetizing a patient, the method comprising inserting a curved cryoneurolysis needle into a target region of a patient, the target region including a target nerve, the curved cryoneurolysis needle having a distal end configured to cool surrounding patient tissue, rotating the curved cryoneurolysis needle to extend alongside the target nerve, and cooling the distal end of the curved cryoneurolysis needle to cause cooling of surrounding patient tissue thus inhibiting the target nerve. In accordance with some forms the curved cryoneurolysis needle comprises one or more imagable portions, and the methods may comprise imaging the one or more imagable portions to confirm placement of the curved cryoneurolysis needle near the target nerve. In certain embodiments the target nerve comprises the medial femoral cutaneous nerve and/or the lateral femoral cutaneous nerve.

Additional embodiments, as well as features and advantages of embodiments of the invention will be apparent from the description herein.

### BRIEF DESCRIPTION OF THE DRAWINGS

FIG. 1 is one embodiment of a method as disclosed herein.

FIG. 2 is a perspective view of one embodiment of a curved cryoneurolysis needle as disclosed herein.

FIG. 3 is a perspective view of one embodiment of a curved cryoneurolysis needle as disclosed herein.

FIG. 4 is a diagram of one embodiment of a method as disclosed herein.

### DESCRIPTION OF THE SELECTED EMBODIMENTS

For the purpose of promoting an understanding of the principles of the disclosed technique, reference will now be made to the embodiments illustrated in the figures and specific language will be used to describe the same. It will nevertheless be understood that no limitation of the scope of the invention is thereby intended. Any alterations and further modifications in the described embodiments, and any further applications of the principles of the invention as described herein are contemplated as would normally occur to one skilled in the art to which the invention relates. One embodiment of the invention is shown in great detail; although it will be apparent to those skilled in the relevant art that some features that are not relevant to the present invention may not be shown for the sake of clarity.

As used here (claims, specification, and other definitions) the following terms have the following meaning:

Articles and phrases such as, “the”, “a”, “an”, “at least one”, and “a first”, “comprising”, “having” and “including” here are not limited to mean only one, but rather are inclusive and open ended to also include, optionally, two or more of such elements and/or other elements. In terms of the meaning of words or terms or phrases herein, literal differences therein are not superfluous and have different meaning, and are not to be synonymous with words or terms or phrases in the same or other claims.

The term “means for” in a claim invokes 35 U.S.C. § 112(f), literally encompassing the recited function and corresponding structure and equivalents thereto. Its absence does not, unless there otherwise is insufficient structure recited for that claim element. Nothing herein or elsewhere restricts the doctrine of equivalents available to the patentee.

The term “and/or” is inclusive here, meaning “and” as well as “or”. For example, “P and/or Q” encompasses, P, Q, and P with Q; and, such “P and/or Q” may include other elements as well.

The term “anesthetizing” as used herein has the meaning, a process for providing temporary loss of sensation, muscle control, and/or awareness.

The term “anterior” as used herein has the meaning, being situated nearest or toward the front of the body. With respect to the spine, the anterior is considered to be the side of the spine closest to the stomach or the throat.

The term “anteriorly” as used herein has the meaning, positioned in a location more anterior with respect to another object where the anterior refers to a position nearer to the front of a reference point.

The term “body portion” as used herein has the meaning, the main or principal part of an object. More specifically, it refers to the part of a vertebra implant that contacts the vertebral body of a neighboring vertebra.

The term “contact” as used herein has the meaning, of two objects the state or condition of physical touching. As used, contact requires at least one location where objects are directly or indirectly touching, with or without any other member(s) material in between.

The term “cranial-caudal orientation” as used herein has the meaning, in the direction along an axis running vertically toward and away from a (standing) patient's skull.

The term “cranial direction” as used herein has the meaning, in the direction toward the patient's head along the cranial-caudal axis.

The term “caudal direction” as used herein has the meaning, in the direction towards the patient's feet along the cranial-caudal axis.

The term “cryoanalgesia” (also known as cryoneurolysis) as used herein has the meaning is the reversible destruction of the signal carrying parts of a nerve. It is a small subset of treatments under the broad umbrella of cryotherapy.

The term “cryo-needle” as used herein has the meaning, a medical device having a slender, usually sharp pointed body and configured to cause freezing at the distal end, (which may include its tip, but also may be portions near to but proximal to the tip), upon activation.

The term “facilitate” as used herein has the meaning, to aid or help accomplish an action or a process to make that action or process easier. The act of facilitation does not need to accomplish the action or process entirely on its own.

The term “front surface” as used herein has the meaning, an exterior surface that may or may not be the anterior most surface.

The term “hole” as used herein has the meaning, a hollow opening within a body, structure, or an object. It can be any shape.

The term “imagable” as used herein has the meaning, a device that is configured to allow for medical imaging of the device during use such as by computed tomography (CT), magnetic resonance (MRI), positron emission tomography (PET), fluoroscopy, and/or ultrasonography. As used herein the “imagable” device is configured to allow a user to visualize all or a portion of the device within the patient during a procedure.

The term “pain block” as used herein has the meaning, inhibition of one or more sensory nerves that relay pain signals to a target region of the patient's body.

The term “intercostal nerve” as used herein has the meaning, the anterior rami of the first 11 thoracic spinal nerves, which are positioned within the intercostal space. Intercostal nerves are peripheral nerves. Each intercostal nerve enters the corresponding intercostal space between the posterior intercostal membrane and the parietal pleura. The nerve then travels forward with the intercostal vessels in the costal groove of the corresponding rib, between the internal and innermost intercostal muscles. The first thoracic nerve divides into a superior part, which joins the brachial plexus, and an inferior part, which becomes the first intercostal nerve. The first six intercostal nerves innervate nearby intercostal muscles, the cutaneous territory on the side of the thoracic wall, the thoracic pleura, and end as terminal branches (anterior cutaneous branch) to innervate the skin near the midline of the chest. The lower intercostal nerves leave their intercostal spaces anteriorly (after giving branches to the intercostal muscles, abdominal peritoneum, and skin) to innervate the anterior abdominal wall, muscles (in particular the rectus abdominis), and the overlying skin. Near their origin, the intercostal nerves send a posterior branch to the paraspinal muscles and the overlying skin, and communicant rami to the sympathetic trunk adjacent to the vertebral column.

The term “local anesthesia” as used herein has the meaning, a medication that causes reversible absence of pain sensation in a target area of the body. When it is used on specific nerve pathways, paralysis also can be achieved.

The term “pain block” as used herein has the meaning, inhibition of one or more sensory nerves that relay pain signals to a target region of the patient's body.

The term “sensory nerve” as used herein has the meaning, a nerve that carries sensory information toward the central nervous system (CNS).

The term “side” as used herein has the meaning, one of the faces on the surface of an object. An object can have multiple faces with a variety of orientations. For example, an object may have a front side, a bottom side, a back side, or a top side.

The term “superficial” as used herein has the meaning, a directional term that indicated one structure is located more externally than another, or closer to the surface of the body.

The term “transverse” as used herein has the meaning, situated or extending across an object or an axis.

The term “withdrawal” as used herein has the meaning, the action of removing or taking away something for a particular location.

With cryo-needle systems, doctors are able to deliver precise, controlled doses of cold temperature only to the targeted nerve through a handheld device. This needle-based procedure is safe, and does not damage or destroy the surrounding tissue. Cryo-needle based treatments use the body's natural response to cold to treat peripheral nerves and immediately reduce pain—without the use of drugs. Treated nerves are temporarily stopped from sending pain signals for a period of time, followed by a restoration of function.

Cryo-needle therapies, work by applying targeted cold to a peripheral nerve, such as an intercostal nerve. A precise cold zone is formed under the skin—cold enough to immediately prevent the nerve from sending pain signals without causing damage to surrounding structures.

The effect on the nerve is temporary, providing pain relief until the nerve regenerates and function is restored.

Cryoneurolysis procedures can include surgeries of the anterior and posterior chest, abdomen, and limbs, with sensory pain relief lasting weeks. In accordance with some modes of practicing the disclosed methods, patients are placed in a lateral decubitus, surgical site up position. In certain embodiments, an intercostal nerve block with local anesthetic is performed before cryoneurolysis. This is done to prevent discomfort that is often associated with cryoneurolysis therapy.

Prior to the present disclosure, cryoneurolysis delivery devices recommend needle insertion at approximately a 30-degree angle. Applicants have discovered that an angled needle insertion followed by perpendicular rotation allows almost any provider to successfully place the tip of the cryoneedle at an optimal location near a target nerve. For example, a 1×99 mm cryoneurolysis needle produces a distal freeze ball 7 mm wide and 17.2 mm in length. Placing a distal needle freeze ball at a 30-45 degree insertion angle on an “anatomic target” with a straight needle in many fascia planes is not optimal because a majority of a cryoneurolysis “freeze zone area” extends retrograde 17.2 mm from the distal tip of the needle. The present disclosure provides cryoneurolysis needles and methods for use of same having an angle bend to maximize the cryoneurolysis freeze zone. The method involves creation of a puncture site followed by introduction of an angled cryoneurolysis needle towards an anatomic target in a horizontal plane. The angled needle is advanced slightly over and beyond the target nerve/area. The cryoneurolysis device is then rotated to a “perpendicular” position allowing a greater horizontal spread. The cryoneurolysis cycle is initiated in this position. This method increases a cryoneurolysis freeze zone area from a distal tip 8 mm to 17.2 mm horizontal spread. Because of the curved needle, when the handheld cryoneurolysis device is rotated to a perpendicular position, the distal tip of the needle is positioned at an angle rotated about 45-90 degrees relative the proximal most portion of the needle. In this way, the distal tip of the cryoneurolysis needle, the portion over which the freeze ball is formed, is positioned alongside the target nerve. As used herein, the term “alongside” refers to the distal portion of the cryoneurolysis needle extending along an axis that is generally parallel an axis along which the target nerve extends. In this way, a greater length of the target nerve is exposed to the freeze ball, which generally extends 10-40 mm, preferably 15-30 mm, even more preferably 15-20 mm along the length of the distal portion of the cryoneurolysis needle.

Postoperative analgesia for the abdominal wall using local anesthetics is possible with sensory blockade of nerves associated with a transversus abdominis plane block (TAP) or a rectus sheath block. The present application provides superficial abdominal wall cryoneurolysis techniques which can provide extended sensory relief compared to local anesthetic techniques. Prior abdominal wall cryoneurolysis methods used a three prong cryoneurolysis needle. Multiple cryoneurolysis cycles had to be performed using the three prong cryoneurolysis needle because the cryoneurolysis freeze width was only 8 mm. In certain embodiments, the present disclosure provides for a single angled cryoneurolysis needle for superficial abdominal wall cryoneurolysis, for example an angled single 1×90 mm cryoneurolysis needle has been utilized. One version for abdominal wall cryoneurolysis would include a cryoneurolysis device having an angled needle introduced starting in a horizontal position and rotation of the device to a perpendicular, starting at a midpoint line from the umbilicus toward the anterior superior iliac spine. In accordance with some forms, an angled cryoneurolysis needle may be utilized to with Superficial TAP and rectus sheath cryoneurolysis methods for all minimal invasive (laparoscopic, Davinci) and open laparotomy incision sites.

Radio frequency ablation of the dorsal rami is a known procedure for chronic back pain. The intended target of such procedures are the nerves that follow a path along the transverse process of a vertebral body. Significant issues can occur if an ablation technique accidentally involves motor nerves, which are deep to the transverse process. Cryoneurolysis of superficial dorsal rami has been shown to be effective for spine fusions using a 3-prong cryoneurolysis needle. This method requires multiple injection sites and procedure time is not always feasible. The angled needle and methods of the present disclosure provide an effective alternative. The present disclosure provides a method for anesthetizing a patient, in particular a patient with chronic back pain, the method comprising guiding a cryoneedle to a target region near a transverse process. Such guiding may be performed utilizing fluoroscopy or ultrasound. The In certain embodiments the methods comprise determining the spatial depth of a cryoneurolysis freeze zone on a distal tip of the cryoneedle. The distal tip cryoneurolysis freeze ball may extend below the treated transverse process. An angled cryoneurolysis needle as provided herein, may be introduced at the target transverse process with cryoneurolysis device rotation perpendicular to the transverse process, such technique provides a horizontal cryoneurolysis freeze zone with linear spread, minimizing the risk to motor nerves deep to the transverse process.

The devices and methods of the present disclosure may be utilized to target intercostal nerves. An angled cryoneurolysis needle may be introduced in a horizontal position near a target intercostal nerve, the needle may then be rotated to a position perpendicular to the target intercostal nerve so as to create a cryoneurolysis freeze zone which is perpendicular to the target nerve. In accordance with some forms a serratus anterior blockade procedure may be performed utilizing an angled cryoneedle as disclosed herein.

Previously, cryoneurolysis for total knee arthroplasty has been advocated using a 3 prong needle. As detailed herein, current 3 prong needles create an 8 mm wide freeze zone. Utilizing a 3 prong needle, multiple one-minute cryoneurolysis cycles may be required for a suprapatellar cryoneurolysis procedure for the medial and lateral femoral cutaneous nerves to be effective. The present disclosure provides, a simplified method comprising a single injection site at a midpoint in the suprapatellar area. In certain embodiments, a single angled cryoneurolysis needle is introduced using horizontal needle placement, and perpendicular device rotation allows for linear horizontal cryoneurolysis freeze zone spread of both the medial and lateral femoral cutaneous nerves through a single site. In some forms, the angled cryoneurolysis needle comprises a 1×90 mm needle. This method reduces cryoneurolysis procedure time for total knee arthroplasty by approximately 50%.

Cryoneurolysis for total hip arthroplasty has also been previously performed utilizing a 3 prong needle. Again, multiple one minute cryoneurolysis cycles (4-5) may be required for a cryoneurolysis procedure of the lateral femoral cutaneous nerves to be effective. The present disclosure provides a simplified method comprising a single injection site. In certain embodiments, a single angled cryoneurolysis needle is introduced using horizontal needle placement and perpendicular device rotation allow linear horizontal cryoneurolysis freeze spread. Such methods may provide horizontal linear spread of the croyoneurolysis freeze zone to lateral femoral cutaneous nerves through a single site. In some forms, the angled cryoneurolysis needle comprises a 1×90 mm needle. This method reduces cryoneurolysis procedure time for total hip arthroplasty by approximately 50%.

In certain embodiments, cryoneurolysis is performed with a single tip cryoneurolysis needle. Placement of the cryoneedle may comprise ultrasound guidance as disclosed herein. In some forms, cryoneurolysis needles are dull and difficult to penetrate the fascial planes of the serratus anterior and the outer intercostal membrane. In certain embodiments, a multipronged cryoneurolysis needle facilitates placement. In certain embodiments, a 3-prong linear array cryoneurolysis needle may be used in conjunction with an angled needle as disclosed herein. Cryoneurolysis with a single long needle requires 1-2 freeze cycles/side at each targeted intercostal space. Cryoneurolysis with a 3-prong needle may require 14-16 freeze cycles per side. In accordance with some forms, a target region is subjected to at least three freeze cycles, preferably 3-20 freeze cycles. In some forms, the target region is subject to five freeze cycles. As used herein a “freeze cycle” is synonymous with “cryoneurolysis cycle” and comprises insertion and cooling of a cryo-needle into patient tissue at a distinct site within a target area.

In certain embodiments the present disclosure provides a method for anesthetizing a human patient undergoing surgery, and/or pain block procedures, the method comprising: sterilizing the patient's skin including a target region, the target region including a site superficial to a target intercostal nerve; inserting at least one cryo-needle into a first tissue region at or near the target site, the cryo-needle having a distal end configured to cool surrounding patient tissue; cooling the distal end of the cryo-needle to cause cooling of surrounding patient tissue thus inhibiting one or more intercostal nerves in the surrounding patient tissue; thereafter, performing surgery on the patient.

In accordance some forms of practicing the disclosed methods, an imagable cryo-needle is used, such that the cryo-needle may be placed using guidance from a medical imaging device. In certain embodiments the angled cryoneedles as disclosed herein comprise one or more imagable portions comprising one or more radiopaque and/or echogenic markers. In some forms, the radiopaque portions comprise iodine containing compounds, barium-containing compounds, gold, tantalum, platinum, tungsten or another heavy metal functioning as a radiopaque agent. In certain embodiments the imagable portions are disposed on a first portion of the angled cryoneedle, the first portion being proximal to the curved portion of the needle such that the imagable portions may be useful to indicate depth of insertion of the needle. Additionally, or alternatively, one or more imagable portions may be positions at or near the distalmost tip of the cryoneedle. In some forms the portion of the needle around which the freeze ball is generated comprises one or more imagable portions. In this way the operator can visualize the placement of the cryoneedle, in particular the freeze zone. Such imagable portions may be present as a band around the circumference of the needle, as markings, or any other suitable arrangement.

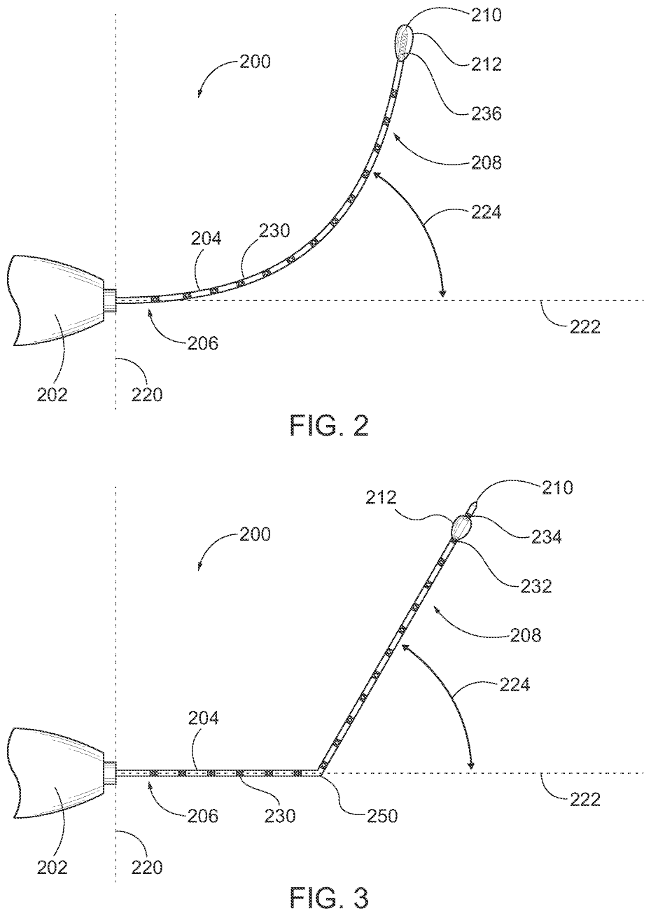

With reference now the embodiment illustrated in FIG. 2, cryoneurolysis device 200, comprising handheld portion 202 and curved needle 204. Curved needle 204 extending from a proximal portion 206 to a distal portion 208 including distal tip 210. Freeze ball 212 is formed on distal portion 208. In the illustrated embodiment, freeze ball 212 is shown as enveloping distal tip 210, it is within the scope of the disclosure to provide a freeze zone spaced from the distal tip sufficient to form a freeze ball along the distal portion but not including the distal tip. Proximal portion 206 extends from handheld portion 202 along first axis 222, approximately perpendicular to second axis 220. Curved needle 204 is curved such that angle 224 is formed between distal portion 208 and first axis 222. The curved needle may adopt any suitable amount of curvature, preferably forming an angle of 45 to 90 degrees. In the illustrated embodiment, the curved needle has an arcuate shape, curving gradually from proximal portion to distal portion. The curved needle comprises imagable portions as described herein, including depth markers 230, and tip marker 236. As illustrated, tip marker 236 is configured such that the entire distal tip, including any portion of the needle configured to form freeze ball 212 is imagable. It is also within the scope of the disclosure to provide a needle with only the distal tip imagable.

Turning now the embodiment illustrated in FIG. 3, cryoneurolysis device 200, comprising handheld portion 202 and curved needle 204. Curved needle 204 extending from a proximal portion 206 to a distal portion 208 including distal tip 210. Freeze ball 212 is formed on distal portion 208. In the illustrated embodiment, freeze ball 212 is shown as spaced from the distal tip sufficient to form a freeze ball along the distal portion but not including the distal tip, it is also within the scope of the invention to provide a freeze zone enveloping distal tip 210, as described above. Proximal portion 206 extends from handheld portion 202 along first axis 222, approximately perpendicular to second axis 220. Curved needle 204 is curved such that angle 224 is formed between distal portion 208 and first axis 222. The curved needle may adopt any suitable amount of curvature, preferably forming an angle of 45 to 90 degrees. In the illustrated embodiment, the curved needle includes bent portion 250 between proximal portion 206 and distal portion 208 which are both linear. The curved needle comprises imagable portions as described herein, including depth markers 230, and tip markers 232 and 234. As illustrated, tip markers 232 and 234 are configured to indicate the freeze zone and the location of freeze ball formation.

In some forms, the present disclosure provides a method of performing cryoneurolysis for abdominal procedures, including hysterectomy procedures. The disclosed method can be used, for example in conjunction with a total hysterectomy, a supracervical or subtotal hysterectomy, or a radical hysterectomy. In accordance with certain embodiments, a local anesthetic, such as bupivacaine, is placed in conjunction with the cryoneurolysis procedure. Ultrasound guided liposomal bupivacaine infiltration into specific intercostal spaces can provide relief for 36-48 hours and avoid pain associated with drains, catheters, and pumps.

Transverse abdominus plant (TAP) block and Rectus sheath blocks using ultrasound and local anesthetics are well documented for pain control for abdominal surgeries. The anterior rami spinal nerves T7-L1 eventually form the ilioinguinal and iliohypogastric nerves and innervate the anterior lateral abdominal wall. The goal of TAP block is to inject local anesthetic in the plane between the internal oblique and transverses abdominal muscles. This will decrease painful stimulation to the abdominal skin, muscles, and parietal peritoneum. TAP blocks do not block visceral pain. TAP blocks can be performed with ultrasound or with knowledge of anatomy.

The rectus sheath is formed by the aponeurosis of the transverse, internal oblique, and external oblique muscles. A rectus sheath block can decrease painful surgical stimulation from the lipoid sternum to almost the pubis. T7-T11 nerves provide sensory to the rectus muscles and skin. The goal of a rectus block is to inject local anesthetic underneath the posterior rectus abdominus sheath where the anterior intercostal nerves enter.

The present disclosure provides systems and methods for modified TAP block procedures including percutaneous cryoneurolysis. Such procedures have been found effective for all laparoscopic, minimally invasive abdominal procedures. Cryoneurolysis using a single or multi-needle device for surgical port, camera incision sites are possible and effective. Cryoneurolysis targeting cutaneous sensory branches from the TAP and Rectus port or incision site innervation are effective methods in reducing painful stimulation. The attached figures, where the blue line indicates surgical port incision examples for cryoneurolysis.

Thus, the present disclosure provides a method for anesthetizing a patient undergoing minimally invasive procedures. In certain embodiments, such minimally invasive procedure includes the placement of a port, or access site for laparoscopic or other access. Such methods may include the step of performing one or more cryoneurolysis cycles targeting sensory nerve branches at or near the port or access site. In accordance with some forms, the minimally invasive procedure comprises a hysterectomy.

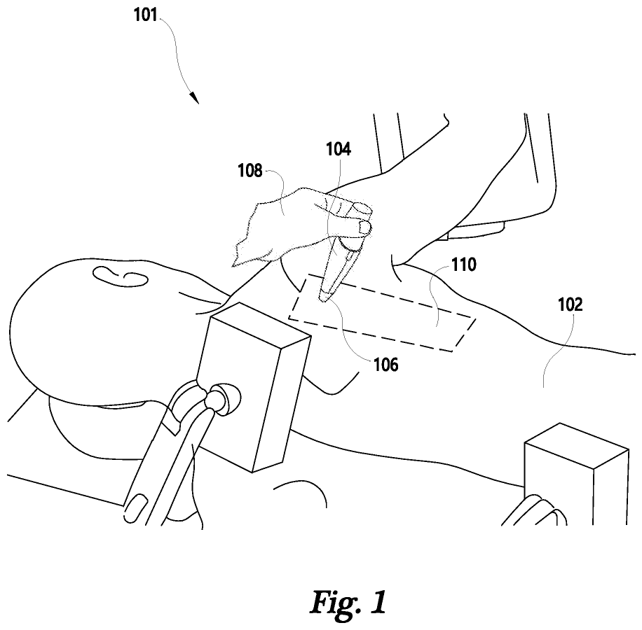

Turning now to a discussion of the illustrated embodiments, FIG. 1 shows one embodiment of a method 101 for anesthetizing a patent. In the illustrated embodiment, the patient 102 is lying in the lateral decubitus position. Target region 110 is identified for cryoneurolysis. In the illustrated embodiment, the target region comprises a posterior mid axillary region extending from about T1 to L5, preferably T7-L1 for a TAP block or T7-T11 for a rectus sheath block. In the illustrated embodiment, a single target region is identified, for example for an abdominal procedure. As discussed herein it is within the scope of the present disclosure to provide bilateral target regions for bilateral anesthetization. An operator 108, for example a surgeon or anesthesiologist, places cryoneurolysis device 104, having a distal end 106 having one or more needles within the target region, and performing one or more cryoneurolysis cycle each comprising inserting the cryo-needle into patient tissue and cooling the distal tip of the cryo-needle.

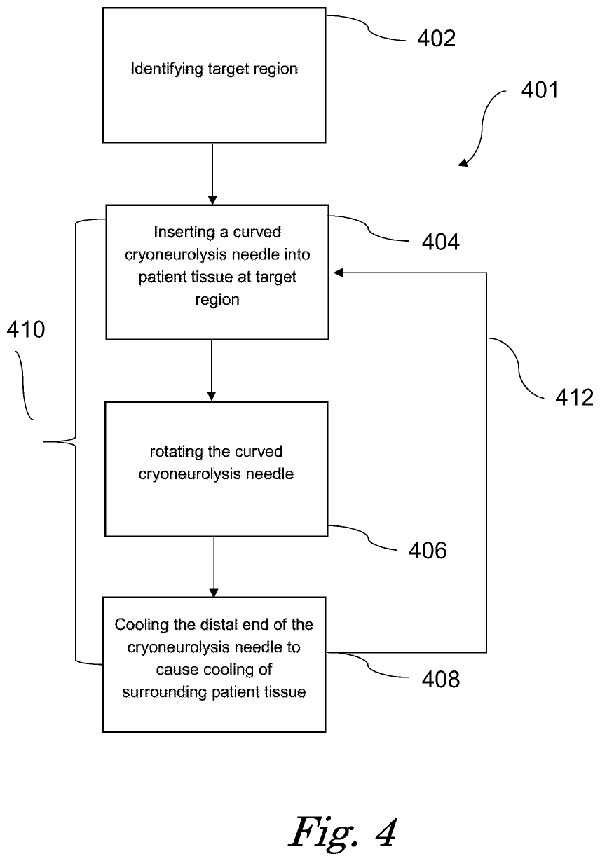

FIG. 4 illustrates one embodiment of a method 401 for anesthetizing a patient as disclosed here. As detailed herein, method of the current disclosure may include further steps, for example administration of a local anesthetic and/or monitored anesthesia care. The illustrated embodiment includes the step of identifying a target region 402. Such target region is preferable associated with one or more sensory nerves. Exemplary target regions as disclosed herein include a posterior mid axillary region at around T1-T8, preferably T2-T6, and or extending from the patient's iliac spine to umbilicus. Additional intercostal nerves may be targeted along their respective lengths at other target regions. The illustrated method includes one or more cryoneurolysis cycles 410. Each cycle includes the step of inserting at least one curved cryoneurolysis needle into patient tissue at the target region 404, rotating the curved cryoneurolysis needle 406, and cooling the distal end of the cryoneurolysis to cause cooling of surrounding patient tissue 408. The cooling cycle 410 may be repeated 412 as needed to effect sufficient cooling of the underlying target nerve. Certain modes of practicing the disclosed methods including bending, or otherwise shaping a cryoneurolysis needle prior to inserting the needle into target region of a patient. Such bending may comprise forming a curved cryoneurolysis needle shape to facilitate use in the above described methods. In this way, a practitioner may obtain a straight or linear shaped needle and form a curved cryoneurolysis needle prior to anesthetizing a patient.

It is within the scope of the present disclosure to provide systems and methods for training the disclosed procedures. For example, in some forms a medical training phantom may be provided having one or more of the intercostal nerves and related anatomy as described herein. It is within the scope of the present disclosure to practice the methods described herein with a training phantom or other analogous training implement. It is also within the scope of the present disclosure to provide training material relating to the performance of the surgical techniques described herein. Such training materials may be in any suitable form, for example written, verbal, or recorded instructing.

The language used in the claims and the written description and in the above definitions is to only have its plain and ordinary meaning, except for terms explicitly defined above. Such plain and ordinary meaning is defined here as inclusive of all consistent dictionary definitions from the most recently published (on the filing date of this document) general purpose Webster's dictionaries and Random House dictionaries.

While the invention has been illustrated and described in detail in the drawings and foregoing description, the same is to be considered as illustrative and not restrictive in character, it being understood that only the preferred embodiment has been shown and described and that all changes, equivalents, and modifications that come within the spirit of the inventions defined by following claims are desired to be protected. All publications, patents, and patent applications cited in this specification are herein incorporated by reference as if each individual publication, patent, or patent application were specifically and individually indicated to be incorporated by reference and set forth in its entirety herein.

What is claimed is:

1. A cryoneurolysis device comprising:
a curved needle having a proximal portion and a distal portion, the curved needle configured to produce a cold zone at or near a distalmost tip of the curved needle for cryoneurolysis of a target nerve.

2. The cryoneurolysis device of claim 1, wherein said curved needle comprises a curved portion between said proximal portion and said distal portion.

3. The cryoneurolysis device of claim 2, wherein said proximal portion is linear.

4. The cryoneurolysis device of claim 3, wherein said distal portion is linear.

5. The cryoneurolysis device of claim 1, wherein said curved needle comprises an arcuate shape extending from said proximal portion to said distal portion.

6. The cryoneurolysis device of claim 1, comprising one or more imagable portions.

7. The cryoneurolysis device of claim 6, wherein said one or more imagable portions positioned to indicate the position of the cold zone produced by said curved needle.

8. The cryoneurolysis device of claim 1, wherein said curved needle is curved to create an angle between said proximal portion and said distal portion, the angle being 45 degrees to 90 degrees.

9. The cryoneurolysis device of claim 1, wherein said curved needle has a length of 90 mm.

10. The cryoneurolysis device of claim 1, wherein said curved needle is configured to create a freeze ball extending at least 15 mm along the length of the needle.

11. A method of anesthetizing a patient, the method comprising:
inserting a curved cryoneurolysis needle into a target region of a patient, the target region including a target nerve, the curved cryoneurolysis needle having a distal end configured to cool surrounding patient tissue;
rotating the curved cryoneurolysis needle to position the distal end alongside the target nerve; and
cooling the distal end of the curved cryoneurolysis needle to cause cooling of surrounding patient tissue thus inhibiting the target nerve.

12. The method of claim 11, wherein the curved cryoneurolysis needle comprises one or more imagable portions.

13. The method of claim 12, comprising:
imaging the one or more imagable portions to confirm placement of the curved cryoneurolysis needle near the target nerve.

14. The method of claim 11, wherein the target nerve comprises the medial femoral cutaneous nerve and/or the lateral femoral cutaneous nerve.

15. The method of claim 11, comprising:
bending a cryoneurolysis needle to form the curved cryoneurolysis needle prior to said inserting.

16. The method of claim 11, wherein the curved needle is curved to create an angle between a proximal portion and the distal portion, the angle being 45 degrees to 90 degrees.
